(19) United States
(12) Patent Application Publication
(10) Pub. No.: US 2021/0137660 A1
(43) Pub. Date: May 13, 2021

(54) TONGUE CLEANING DEVICE WITH MEDICINAL RESERVOIR

(71) Applicant: Bio-Life Innovations, LLC, North Canton, OH (US)

(72) Inventor: David J. Boord, North Canton, OH (US)

(21) Appl. No.: 17/155,516

(22) Filed: Jan. 22, 2021

Related U.S. Application Data
(63) Continuation-in-part of application No. 16/261,237, filed on Jan. 29, 2019.
Application No. 16/261,237 is a continuation-in-part of application No. 15/012,520, filed on Feb. 1, 2016, now Pat. No. 10,188,414.
(60) Provisional application No. 62/964,575, filed on Jan. 22, 2020.
(60) Provisional application No. 62/988,343, filed on Mar. 11, 2020.
(60) Provisional application No. 62/989,441, filed on Mar. 13, 2020.
(60) Provisional application No. 62/110,509, filed on Jan. 31, 2015.
(60) Provisional application No. 62/264,760, filed on Dec. 8, 2015.

(51) Int. Cl. A61C 17/02; A61B 17/24; A46B 15/00; A46B 11/00

(52) U.S. Cl. CPC A61C 17/0208; A46B 11/001; A46B 15/0081; A61B 17/244

(57) ABSTRACT

Provided is a tongue cleaning device that can be attached and detached from a typical vacuum flow generating tool provided in dental examining rooms, surgical suites, and hospital rooms. A small amount of medication may be stored in and dispensed from a head of the tongue cleaning device while a trough may be configured to collect substances thereon and vacuumed through a cavity defined within a handle of the device. The device may be configured to release the medication against the tongue of a patient by capillary action and through one or more dispensing vents, scrape against the surface to clean the tongue, and vacuum out the debris through the body of the device.

### CROSS-REFERENCE TO RELATED APPLICATIONS

This application claims priority to and the benefit of U.S. Provisional Patent Application No. 62/964,575 entitled “Tongue Cleaning Device” filed on Jan. 22, 2020, U.S. Provisional Patent Application No. 62/988,343 entitled “Tongue Cleaning Device” filed on Mar. 11, 2020, and U.S. Provisional Patent Application No. 62/989,441 entitled “Tongue Cleaning Device Insert Chamber” filed on Mar. 13, 2020, all of which are hereby incorporated by reference in their entireties.

This application is also a continuation-in-part of U.S. patent application Ser. No. 16/261,237 entitled “Tongue Cleaning Device” filed on Jan. 29, 2019 which is a continuation-in-part of U.S. patent application Ser. No. 15/012,520 (now U.S. Pat. No. 10,188,414) filed on Feb. 1, 2016 and claims priority to U.S. Provisional Patent Application No. 62/110,509 entitled “Tongue Cleaning Device” filed on Jan. 31, 2015 and U.S. Provisional Patent Application No. 62/264,760 entitled “Tongue Cleaning Device” filed on Dec. 8, 2015, each of which are incorporated by reference in their entireties.

### FIELD OF THE INVENTION

The present disclosure generally relates to tongue cleaning devices and, more particularly, to a tongue cleaning device used in cooperation with a vacuum to remove tongue debris from the tool after the tool removes the debris from the tongue. The present disclosure also generally relates to tongue cleaning devices that is configured to deposit a liquid solution on the tongue during the cleaning process.

### BACKGROUND

Numerous types of tongue cleaning devices are known in the art. All have advantages and disadvantages. The art generally desires a tongue cleaning device that effectively cleans the plaque and debris from the tongue while being safe, easy to use, and effective.

One drawback with existing tongue cleaning devices is that the debris collected by the device can fall off of the device shortly after the device is removed from the tongue. Most home users clean their tongues over a bathroom sink and the debris retention problem does not bother them or stop them from using the devices. In other settings, the debris retention problem is of more concern. One such setting is a hospital where it is becoming more desirable to clean the tongues of bedridden patients in order to reduce the amount of bacteria of the patient's tongue. Another situation is a surgical suite wherein the tongue of a patient is cleaned prior to anesthesia. Those who use tongue cleaning devices in these situations desire a tongue cleaning device that will retain or remove the debris from the scraping wall of the tongue cleaning device so that the tongue cleaning device may be easily used on a patient who is lying on his back.

### SUMMARY OF THE INVENTION

The disclosure provides a vacuum tongue cleaning device that can be attached and detached from a typical vacuum flow generating tool provided in dental examining rooms, surgical suites, and hospital rooms. The vacuum tongue cleaning device also can be used with a portable vacuum flow generating device for use in nursing home rooms or by home health care providers. A vacuum port may be provided at the head of the device into a vacuum reservoir and may enable suction therethrough. A vacuum port may be provided through the body, to further transfer debris and a vent opening or openings may be provided on the top or side of the body of the device to facilitate different levels of suction. For instance, the more the vent opening(s) may be closed, as by a finger, the more suction that may be provided to the vacuum port at the head of the device.

The device may include a vented portion. The vented portion may be molded with the body or the vented portion may be an insert relative to the body that is positionable against the body. In both embodiments of the vented portion, a small amount of medication may be stored behind the vented tip in a medicinal reservoir. The device may be configured to release the medication against the tongue of a patient by capillary action and through one or more dispensing vents, scrape against the surface to clean the tongue, and vacuum out the debris through the body of the device.

In one embodiment, provided is a tongue cleaning device, comprising an elongated body including a head and a handle, including a vacuum passage that extends therein, wherein the vacuum passage facilitates suction of debris through the elongated body; a working surface on a first side of the head, the working surface including an inlet that is fluidly connected to the vacuum passage and that is adjacent a working edge, wherein the working edge is configured to scrape a surface of the tongue a trough adjacent the working edge and in communication with the inlet. A reservoir may be positioned on the head distal to or away from the inlet that including a plurality of dispensing vents wherein the reservoir is configured to hold a substance that configured to be released through the dispensing vents when in use.

The substance may be an antibacterial agent, a medicine, or a freshener agent or the substance is chlorhexidine gluconate, carbamide peroxide, a mixture of hydrogen peroxide and water. The inlet may be aligned on an axis defined by the handle and may face away from the handle. A wall may separate the inlet from the reservoir and extend from the head a direction that is generally normal to the axis defined by the handle. The device may further include an inlet that is fluidly connected to the opening and the vacuum passage wherein the inlet is opposite the wall and across the opening. The working edge may be configured to direct debris into the opening. A brush member may be provided on a second side of the head, wherein the brush includes a plurality of bristles or brush teeth that may be integral to a brush plate.

In another embodiment, provided is a tongue cleaning device comprising a first elongated portion that includes a head portion and a handle portion and a second elongated portion that includes a head portion and a handle portion wherein the first elongated portion is attachable to the second elongated portion to form a head in communication with a cavity defined within a handle wherein the head includes a reservoir configured to dispense a substance and a trough configured to collect a substance to be vacuumed through the cavity. The handle portion of the first elongated portion may includes a first portion of the cavity and the head portion of the first elongated portion includes a plurality of dispensing vents and a trough aperture. The handle portion of the second elongated portion may include a second portion of a cavity and the head portion includes a reservoir and a trough. An inlet may be provided between the first elongated portion and the second elongated portion to allow the trough to communicate with the cavity. The plurality of dispensing vents may be configured to release a substance from the reservoir. A wall may be positioned between the reservoir and the trough such that the trough is configured to collect material from a surface of the tongue to be withdrawn through the cavity. The first elongated portion and the second elongated portion may be attached along an elongated axis that is generally parallel to the axis of the cavity. The first elongated portion and the second elongated portion may be formed of a common continuous material.

### BRIEF DESCRIPTION OF THE DRAWINGS

The present teachings may be better understood by reference to the following detailed description taken in connection with the following illustrations, wherein:

FIG. 1 is a perspective view of an embodiment of a tongue cleaning device connected to a vacuum source;

FIG. 2 is a side section view taken along the longitudinal axis of the device of FIG. 1;

FIG. 3 is an enlarged view of the encircled portion of FIG. 2;

FIG. 4 is an enlarged bottom view of the working head of the tongue cleaning device of the FIG. 1 configuration;

FIG. 5 is a top plan view of another embodiment for the tongue cleaning device of this disclosure;

FIG. 6 is a section view taken along line 9-9 of FIG. 5;

FIG. 7 is an enlarged view of the encircled portion of FIG. 6;

FIG. 8 is a perspective view of the bottom of the working head of the tongue cleaning device of FIG. 5;

FIG. 9 is a top plan view of another configuration of the vacuum control defined by the handle;

FIG. 10 is a perspective view of an embodiment of the tongue cleaning device of the instant application;

FIG. 11A is a top view of the tongue cleaning device of FIG. 10;

FIG. 11B is a bottom view of the tongue cleaning device of FIG. 10;

FIG. 11C is an enlarged view of the tongue cleaning device of FIG. 10;

FIG. 12 is a top exploded perspective view of the tongue cleaning device of FIG. 10;

FIG. 13 is a bottom split view of the tongue cleaning device of FIG. 10;

FIG. 14 is an enlarged bottom view of the tongue cleaning device of FIG. 10;

FIG. 15 is an enlarged split bottom view of the tongue cleaning device of FIG. 10;

FIG. 16A is an image of another embodiment of the tongue cleaning device including a brush head according to the instant disclosure;

FIG. 16B is a rear perspective view of another embodiment of the tongue cleaning device including a brush head according to the instant disclosure;

FIG. 16C is a side view of the tongue cleaning device of FIG. 16B;

FIG. 16D is a rear perspective view of the tongue cleaning device of FIG. 16B;

FIG. 17A is a side view of an embodiment of a tongue cleaning device;

FIG. 17B is a side view of an embodiment of the tongue cleaning device;

FIG. 18A is a front view of an embodiment of a tongue cleaning device and FIG. 18B is a back view of an embodiment of a tongue cleaning device;

FIG. 19A is a perspective view of an embodiment of the tongue cleaning tool with the material recess at the front of the working head sealed with a removable seal;

FIG. 19B is an enlarged view of the working head of FIG. 19A;

FIG. 19C is a top plan view of the tool of FIG. 19A;

FIG. 19D is a section view taken along line A-A of FIG. 19C;

FIG. 19E is a side elevation view, partially in section, of the tool of FIG. 19A;

FIG. 19F is a bottom plan view of the tool of FIG. 19A;

FIG. 20A is an exploded perspective view of the tool of FIG. 19A;

FIG. 20B is an enlarged cross section view of the front of an embodiment of the head;

FIG. 21 is an enlarged bottom view of the head of an embodiment of the tongue cleaning tool;

FIG. 22 is an enlarged cross section view of the head of an embodiment of the tongue cleaning tool;

FIG. 23 is a perspective view of an embodiment of a material holder for the tongue cleaning tool;

FIG. 24 is an exploded perspective view of an embodiment of a material holder and tongue cleaning tool; and

FIG. 25 is a perspective view, partially in section, of an embodiment of the tongue cleaning tool.

### DETAILED DESCRIPTION

Reference will now be made in detail to various embodiments of the present teachings, examples of which are illustrated in the accompanying drawings. It is to be understood that other embodiments may be utilized and structural and functional changes may be made without departing from the respective scope of the present teachings. Moreover, features of the various embodiments may be combined or altered with other embodiments without departing from the scope of the present teachings. As such, the following description is presented by way of illustration only and should not limit in any way the various alternatives and modifications that may be made to the illustrated embodiments and still be within the spirit and scope of the present teachings. In this disclosure, numerous specific details provide a thorough understanding of the subject disclosure. It should be understood that aspects of this disclosure may be practiced with other embodiments not necessarily including all aspects described herein, etc.

As used herein, the words “example” and “exemplary” means an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

Turning to the drawings, shown are various configurations of a tongue cleaning device 2. Each device 2 has a body that defines a passage configured to direct a vacuum flow to a working edge to remove debris from the tool after the tool has removed debris from the tongue. In some of the exemplary configurations, a liquid or gel such as an antibacterial material can be applied to the tongue immediately after, before, or during the cleaning by the working edge. The liquid or gel being applied can be chlorhexidine gluconate, carbamide peroxide, a mixture of hydrogen peroxide and water, another suitable material that functions as a disinfectant or an antiseptic, or any other desired substance that may provide a desired effect. For example, the applied substance may have cleaning, disinfecting, antiseptic, whitening, breath freshening, numbing, etc. properties. The applied substance may also include personalized prescription medicine for the user or the like.

The device 2 may be used for ICU patients, pre-op surgical patients prior to intubation, and higher risk in-patient oral care. Variations of device 2 may also be used in dental offices, oral surgery offices, and orthodontic offices for surgical and non-surgical care. Variations of device 2 may also be used in nursing homes, during in-home health care, and the like. Device 2 may also be used as and replace a bedside suction device to suction oral secretions.

In the following description and drawings, the downward or lower direction is the direction disposed towards (90 degrees to the surface being cleaned) the tongue and the upward direction is disposed directly away from the tongue when the tool is engaging the tongue. The rear of the device is towards the handle while the front is toward the tip of the head. Each configuration of device 2 can be fabricated from any of a variety of rigid materials such as moldable or printable plastics, medical device polymers, ceramics, and/or metals. Each device 2 can be fabricated from titanium. Device 2 can be fabricated from a material that can be sterilized under high heat. The device 2 may be predominately used in conjunction with suction to remove any debris loosened by the working edge and/or brushing motion and to remove the applied substance soon after application or release to prevent acute respiratory distress syndrome (ARDS), which is a life-threatening lung injury that results from fluid leaking into the lungs.

Each exemplary configuration of device 2 generally includes a handle 4 and a head 6. Handle 4 includes a first end 3 disposed at head 6 and a second end 5 configured to be selectively connected to a vacuum flow generator or vacuum source 8. Vacuum source 8 can be provided in any or a plurality of different ways known in the art. Vacuum source 8 can be a manually-powered squeeze bulb, a manual or powered pump, an electrically-powered vacuum pump typically used in a surgical suite or a dentist's office that includes an air-liquid separator and a waste reservoir to gather liquid and debris, or a source of vacuum flow otherwise powered manually or remotely.

Vacuum source 8 may be operatively and removably connected to the second end 5 of handle 4 with a connection that is substantially airtight so that the vacuum flow does not leak at the connection and so that full suction is provided to through the device 2 and up to the head 6 of the device 2. The connection may be releasable and removable so that device 2 can be readily removed after use and disposed of (in the case that device 2 is a single-use item) or cleaned (in the case that device 2 is not a single use item). The removability of the connection allows a single vacuum source 8 to be used with multiple patients by connecting new and clean devices 2 as the vacuum source 8 is moved from patient to patient, without having to clean or switch out the vacuum source 8 itself.

In an embodiment of device 2, shown in FIG. 1 for example, the second end 5 of handle 4 is in the shape of a hollow cylinder with one end 10 slightly tapered or having a smaller diameter and the other end bounded by a rounded flange or portion with a larger diameter 12. The tapered end 10 allows device 2 to be slid into the end of a vacuum tube 14 and flange 12 creates a stop for the vacuum tube 14. As shown in FIG. 10, the flange 12 may be of a greater diameter than the tapered end 10 and that greater diameter may also generally extend to the remaining body of the device 2, i.e., the flange 12 may protrude from the rest of device 2 as shown in FIG. 1 or the flange 12 may not protrude from the rest of device 2, but may protrude only in respect to the tapered end 10 as shown more similarly in FIG. 10. It is noted that the opposite configuration may also be used wherein the end of the vacuum tube 14 is of a smaller or tapered diameter so as to be inserted into the end 10 of the device 2. The device 2 may then include a flange 12 or other stop point on its interior to facilitate positioning of the vacuum tube 14 therein.

Device 2 (and end 10 thereof) may be secured to the vacuum source 8 or end of a vacuum tube 14 by friction and by the vacuum force applied by the vacuum tube 14. Device 2 (and end 10 thereof) may also be secured to the vacuum source 8 or end of a vacuum tube 14 by any other suitable means including a threading, snap fit, pressure fit, fasteners, a locking mechanism including mating recessed and protruding portions that, for example, could be spring-loaded and releasable by a button, a bayonet connection, and the like.

Handle 4 may define or comprise therein a vacuum passage 16 that directs a vacuum flow supplied by vacuum source 8 through the body of device 2. A backflow preventer valve 18 is optionally provided along vacuum passage 16 to minimize the risk that vacuumed saliva and debris present in vacuum tube 14 would flow back into a newly-installed device 2. As stated herein, this may allow flexibility in using multiple sequential or tandem devices 2 with the same vacuum source 8 without having to clean or modify the vacuum source 8 in between uses with subsequent devices 2. A biased ball check valve 18 is depicted in FIG. 2 but resilient flexible flap valve or other backflow prevention device can also be used and is contemplated herein.

A primary vacuum control opening 20 is defined by a portion of handle 4. As shown in FIG. 1, primary vacuum control opening 20 can be defined by the upper surface portion of device 2 such that a user's index finger or thumb can be positioned over at least a portion of vacuum control opening 20 when the user is holding device 2 in the same manner as one holds a knife, fork, or pencil when pushing down with an index finger or thumb. In this case, the index finger or thumb of the user can push down on device 2 but can also control the strength of the vacuum applied to head 6. For example, when the user wants a stronger vacuum force applied to head 6, the user covers the entire vacuum control 20 opening with their finger so that the full force of the vacuum source 8 is pushed through the vacuum passage 16 and to the head 6 of the device 2. When the user wishes to lessen the vacuum force, the index finger can slide to expose portion of vacuum control opening 20 to allow some of the vacuum force from the vacuum source 8 to be delivered to and released through the vacuum control opening 20 instead of continuing fully through the vacuum passage 16 and to the head 6 of the device 2. The device 2 may also include indented or recessed areas 23 on the backside surface portion of device 2 to accommodate the user's thumb and/or middle finger to provide a more ergonomic shape and use of the device 2, see FIG. 11B for example.

The particular shape of the vacuum control opening 20, e.g. larger elongated slit, multiple smaller apertures, etc., are further described herein. A secondary vacuum control opening 21 can also be provided in front of vacuum control opening 20 or at another location on head 6 or handle 4 to ensure the vacuum flow through handle will always have an unsealed vacuum inlet in the unlikely occurrence that head 6 is pushed down to where it could vacuum seal to the tongue. As long as secondary control opening 21 is not covered by the user, device 2 cannot entirely vacuum seal to the tongue.

In the embodiments of FIGS. 1-4, vacuum control opening 20 is tear drop-shaped. The tear drop-shape may provide a wider range of control than would be provided by a control opening shape having a constant width such as the vacuum control opening 20 depicted in FIGS. 5-6. Positioning the narrow end of the tear drop-shaped vacuum control opening 20 closest to the palm of the user's hand (the second end 5 of the handle 4) allows a slight amount of vacuum pressure to be released by sliding the user's index finger forward towards head 6. This configuration ensures the user retains good control over head 6 as well having good control over the vacuum source. In this configuration, secondary vacuum control opening 21 is depicted through the top of handle 4 but it can be located anywhere along handle 4. Unlike FIGS. 5-6 which show a control opening shape 20 having a constant width that may be considered generally rectangular, FIGS. 10 and 13, for example, show a control opening shape 20 that is tapered at both ends as the shape extends toward the first end 3 and second end 5 of the handle 4. In this embodiment, the control opening shape 20 may be considered generally ovular. The tapered ends of the ovular shape of the control opening 20 in this embodiment may be less tapered than the tapered end of the tear drop shape 20 depicted in FIG. 1. In any event, the shape of the control opening 20 may be slightly smaller in width than the average width of a user's fingertip and may extend in length to an area that is approximately the same or slightly longer than an average user's finger pad. The ovular control opening 20 may also be substantially thinner than the user's finger and thus easy to find and seal with the finger. The edges of the control opening 20 may be slightly rounded or tapered, so as to facilitate a seal of the vacuum and comfort to the user.

In one embodiment as illustrated by FIG. 9, a plurality of vacuum control openings 20 may be defined along the longitudinal axis of the handle. These openings 20 may be disposed close together (such as providing a plurality of openings disposed within a half inch space) so that a user's finger can cover the plurality of openings 20 and then be slid in one direction or another to expose one opening at a time or a plurality of openings 20 at a time to relieve vacuum force. The openings can be the same size or different sizes. In the configuration of FIG. 9, the openings are smaller close to the head 6 or first end 3 of the handle 4 and progressively get larger toward the second end 5 of the handle 4. For example, the opening closest to head 6 may be 1 mm in diameter while the opening 20 farthest from head 6 can have a diameter of 4 mm with the plurality of openings 20 between the two ends being evenly sized. In the example with five openings, the second opening has a diameter of 1.75 mm, the middle opening diameter is 2.5 mm, and the fourth opening is 3.25 mm. It is noted that secondary vacuum control opening 21 can also be disposed on the front, side, or bottom of handle 4.

In one embodiment as illustrated by FIGS. 5-6, a single opening 20 may be disposed on the side of handle 4 and could be controlled by the user's middle finger. The opening 20 may be provided in the form of an elongated slit, such as that shown in FIGS. 1-2. The slit can be about 8 to 12 mm long and 1 to 4 mm wide. This slit allows the user to control the vacuum delivered to the head 6 of device 2. A benefit with the slit is that it is substantially thinner than the user's finger and thus easy to find and seal with the finger.

In another embodiment, a sliding cover may be provided to selectively close vacuum control opening 20. Sliding cover may slid back and forth by the user's finger to open and close opening 20 and may generally stay in the set configuration until additional force by the user's finger is applied to move the cover. An advantage with cover is that the user can change the position of his fingers without changing the vacuum force. A locking tooth and recess may be used hold the cover in place until intentionally moved by the user. The locking tooth may be carried by the cover and the recess in the handle 4 or the locking tooth can be carried by the handle 4 and the recess on the cover to facilitate mating.

In the embodiments depicted in FIGS. 1-3 and 5-6, as well as FIGS. 10 and 19, the longitudinal axis of the handle is substantially straight. In another configuration, a portion of handle 4 may be curved when viewed from the side. Using a generally curved handle allows the working head of device 2 to easily reach the rear of the tongue especially when the user's mouth is not fully open or when other implements are disposed in the mouth (such as a breathing tube). The curvature, and radius thereof, can be in a range of three to twelve inches.

In an embodiment, handle 4 may have an enlarged grip area 26 that defines opening/slit 20 or openings 20. Grip area 26 defines three recesses 28 that receive the user's thumb and two fingers for added control over device 2. Opening/slit 20 or openings 20 may be defined by handle 4 within one or more of these recesses 28 which helps the user's finger to selectively cover openings 20 to control the vacuum. As shown in the drawings, opening 20 or the group of openings 20 need not be centered within recess 28. Instead, the opening 20 or the group of openings 20 are positioned forward toward head 6 such that the user can grip the rear of recesses 28 without covering opening 20 or openings 20. As such, each recess 28 has a forward portion and a rear portion with at least one of recess 28 defining opening 20 in at least the forward portion of recess 28. In some of the configuration, the entire opening 20 can be in the forward portion of recess 28. In other configuration, opening 20 can extend into the rear portion.

The vacuum flow applied to head 6 can have a measured vacuum strength from about 0.25″ to 7.00″ Hg Gauge vacuum at standard temperature and pressure. About 1.00″ to 2.00″ Hg Gauge vacuum is a desirable range. As such, device 2 can be configured with vacuum control opening 20 and secondary vacuum control opening 21 to cooperate to provide for a minimum of 0.25″ Hg applied to head 6 when openings 20 and 21 are completely uncovered even when a vacuum pressure of 7.00″ Hg is applied to handle end 10. When opening 20 is completely covered, most of the 7.00″ Hg vacuum pressure is applied to head 6 with opening 21 providing a safety against vacuum sealing head 6 to the tongue.

In the exemplary configurations, head 6 is integrally connected to the first end 3 of the handle 4 and vacuum passage 16 of the handle extends into head 6. Head 6 tapers outwardly from point 30 (at or near first end 3) having a width equal to the portion of handle 4 where head 6 joins with handle 4 to a middle portion defining a maximum width 32. The maximum width location may be disposed intermediate the inlet 40 to vacuum passage 16 and the first end 3 of handle 4. This is so that inlet 40 is forwardly positioned within a closed head. The outermost end 42 of head 6 is smoothly rounded between these two maximum width dimension points 32. Compared between FIGS. 4 and 11B, the degree of tapering of the head 6 from point 30, to the maximum width 32, and to the outermost end 42 may vary. For example, the device 2 in FIG. 11B may have a longer area between point 30 and outermost end 42, the maximum width 32 may be smaller, and/or the maximum width 32 may occur at a point that is closer to either point 30 or outermost end 42. Additionally, as shown in FIG. 11B, the maximum width 32 may (instead of the “point” shown in FIG. 4), may extend a distance on the head 6 of the device 2 so that the device may have a substantially straight edge at this maximum width 32 along an opposite head surface 6′ on the top of the head 6 as illustrated by FIG. 11A. In an embodiment, the head surface 6′ may be substantially planar in shape that is sufficiently sized to accommodate the trough 60, wall 54 with working edge 50, recess 90 or reservoir 210 and dispensing vents 220 as well as be sized to shape within the mouth of a user. For example, in FIG. 11B, the length of the maximum point 32 extends the length of trough 60. Moreover, the outermost end 42 and rounded tip may vary between embodiments, such that the tip may be more or less rounded.

Head 6 may include a working edge 50 defined at the junction or corner of a bottom surface 52 of a scraping wall 54 and an inner surface 56 of wall 54. This corner is rounded to avoid damaging the tongue. Exemplary radii for this working edge are disclosed in U.S. Pat. No. 7,029,484 which is incorporated herein by reference. When working to remove debris from the tongue, working edge 50 engages the tongue and inner surface 56 extends upwardly from the tongue. Debris and saliva are gathered on inner surface 56 when working edge 50 engages the tongue surface and moved along the tongue surface with some downward pressure. Working edge 50 and inner surface 56 extend along a curve that substantially defines a half circle (when viewed from below) from middle portion 32 on one side of head 6 to middle portion 32 on the other side of head 6. Working edge 50 and inner surface 56 define an arc of 120 degrees to 180 degrees when viewed from the bottom of device 2 as shown in FIGS. 4 and 8. The rear ends of working edge 50 are thus disposed behind inlet 40.

Working edge 50 is disposed lower than the bottom of the central portion of head 6 and the front half or head-end of handle 4 (when handle 4 is horizontal) as shown in FIGS. 3 and 7 wherein working edge 50 is stepped down (at reference numeral 58) from head 6 to allow working edge 50 to engage the tongue free of interference from head 6 and handle 4. Working edge 50 can have a flat lower surface or a curved lower surface shaped to engage the tongue such as the concave configuration depicted in FIG. 26 of U.S. Pat. No. 7,029,484 or a convex configuration wherein scraping wall 54 has a maximum height at outer or front end 42 of head 6. Working edge 50 can be angled when viewed from the side such that the center portion of working edge 50 is disposed lower than its rear ends. Working edge 50 can be defined to match the shape of a tongue with dual curves. Working edge 50 may include a curved shape that follows the rounded edge of device 2 and that follows the curved shape of the medicinal reservoir 210 and dispensing vents 220.

Head 6 defines a trough 60 disposed along a majority of inner surface 56 which is disposed above working edge 50. Trough 60 is disposed above inner surface 56 and has a defined width and depth. The defined width helps device 2 function at different angles because a portion of the vacuum flow is delivered through trough 60 regardless of the angle of head 6. A deeper trough 60 allows for better containment of debris while a shallower trough 60 provides for a wider range of approach. Trough 60 directs the vacuum flow to inner surface 56 and allows the mix of debris and saliva to be efficiently removed from inner surface 56 by the vacuum flow through device 2.

Trough 60 is defined by a curved top wall 62 that smoothly joins with inner surface 56 free of any steps or ledges where the vacuum flow would be interrupted or where debris would gather. Curved top wall 62 can be provided in a front curved quarter, a central flat portion, and a rear curved quarter. In one configuration, the top of wall 62 can be disposed higher than the uppermost portion of vacuum inlet 40. In another configuration, a portion of vacuum inlet 40 is defined by top wall 62. The ends 64 of trough 60 can be smoothly rounded. Ends 64 are disposed behind inlet 40. Trough 60 is also partially defined by a rear surface 66 disposed substantially concentric with inner surface 56 to define the width of trough 60. Rear surface 66 extends between ends 64 and along the rear of each end until reaching inner surface 56. Rear surface 66 is interrupted by inlet 40. Rear surface 66 can be vertical and parallel with inner surface 56 or can be angled towards the rear of device 2. Rear surface 66 may not extend down as far as inner surface 56 but may function to limit leakage of the vacuum from the rear of head 6. Rear surface 66 is defined by a central wall 70 of head 6. Rear surface 66 defines the constant width of trough 60.

Central wall 70 defines inlet 40. As shown in the FIGS. 4, 11B, and 11C, inlet 40 is defined by portions of rear surface 66 as well as top wall 62 of trough 60. Inlet 40 also has a portion that opens through the bottom surface 72 of central wall 70. This configuration distributes the vacuum flow evenly throughout trough 60 and provides for some vacuum under the central portion of head 6. Surface 72 prevents the vacuum from being delivered to an entirely open lower surface of head 6. Extending inlet 40 through top wall 62 delivers a portion of the vacuum flow directly to the top curved wall of trough 60 which allows the vacuum to be delivered quickly to inner surface 56 with a minimum of turns and obstacles. Bottom surface 72 is disposed above working edge 50 to minimize the likelihood device 2 will become stuck to the tongue when in use.

Head 6 defines an inlet tube 80 between inlet 40 and the end of vacuum passage 16. In order to decrease the chance of clogging and in order to increase the removal efficiency of device 2, inlet tube 80 may include throttle 82 which is an area of decreased cross sectional area in order to speed up the vacuum flow. A plurality of throttles 82 can be used. In this configuration, inlet tube 80 increases the speed of the vacuum flow after the flow enters inlet 40. The speed is then decreased prior to entering vacuum passage 16. The decrease in area is substantial and is forty to sixty percent in the exemplary configuration. The transitions can be abrupt in order to interrupt laminar flow. In the exemplary configuration, a pair of chambers define inlet tube 80 with the smallest opening 82 of the vacuum passage being defined between the two chambers. The outermost chamber that defines inlet 40 allows debris and saliva to be gathered prior to being subjected to increase in velocity through the necked down section between the two chambers. In other configurations, a single chamber can be used. In other configurations, the transitions can be smooth in order to maintain laminar flow. The increase in the speed of the flow helps lift debris and saliva up into the vacuum passage when device 2 is in use.

It is noted that in an alternative embodiment, inlet 40 may be located at trough end 64. Inlet 40 can be located at one trough end 64, at both trough ends 64, and these can be used with or without the centrally-located inlet 40. Branch vacuum pathways 83 are defined by central wall 70 and are in fluid communication with vacuum passage 16. Locating inlets 40 at ends 64 helps remove liquid from the edges of the tongue. Inlets 40 can be disposed through top wall 62, rear surface 66, ends 64 or a combination of these surfaces.

In the configuration of FIGS. 5-8 and 10-25, device 2 includes the ability to distribute a substance such as a liquid or gel antibacterial agent, a medicine, or a freshener agent to the tongue. For example, in FIGS. 5-8, shown is a fluid distribution channel 84 defined by head 6 that may apply the substance directly after working edge 50 is pulled from a rear portion toward a front portion of the tongue. Channel 84 extends from the top of head 6 to the front of head 6 along the curve of the front wall of head 6. The channel 84 can be used to distribute chlorhexidine to the user's tongue immediately after the surface of the tongue is scraped. Other antibacterial solutions can be applied. Channel 84 can be a single channel with a single outlet 86 defined by bottom surface 52 of scraping wall 54. Channel 84 can be branched into a plurality of branches or can define a common plenum with a plurality of outlets 86 spaced along bottom surface 52 of wall 54. With outlets 86 defined by bottom surface 52, the liquid is directly applied to the tongue surface to minimize waste. The direct application also minimizes any squirting into the throat which can cause gagging. Locating outlets 86 forward of working edge 50 is desired so that the applied material is not immediately scraped up and then vacuumed by device 2. In another configuration, outlets 86 can be defined by the outer front surface 88 of wall 54 with the outlets angled down to apply liquid directed through the outlets down onto the tongue.

In an embodiment, the head 6 may include an elongated port or recess 90 for distributing material therefrom. In the embodiment of FIGS. 5-8 the elongated port 90 may removably receives a tube from a supply 92 of the material to be distributed through channel 84. In the embodiment of FIGS. 10-18B, the head 6 may include a reservoir 210 with vents 220 (FIGS. 10-15) or a recess 90 to retain a material holder 192 therein (FIGS. 19A-25). This disclosure contemplates that the device 2 may include both a recess 90 or reservoir 210 with vents 220 that may be integrally formed, made by attaching to elongated halves together, or the recess includes a separate material holder or insert 192 that is configured to store, transport, and dispense a liquid or material as well as a separate trough 60 to collect the liquid after it may be spread within the mouth or surfaces within the mouth. The resulting mix of dispensed liquid and saliva or water within the mouth may be further applied with the brush 310 positioned along the head of the same device 2, collected by a scraping wall into the trough 60 and then promptly evacuated through the cavity in the handle. This disclosure contemplates that any combination of the features described in the respective embodiments or Figures herein may be individually combinable and this disclosure is not limited.

In the embodiment of FIGS. 5-8, the user may connect a supply 92 (such as a bottle or a supply tube connected to a pressurized supply of fluid) of the material to port 90 when application of the material is desired. Supply can be carried by handle 4, can be carried by the user, or can be an independent supply having a pump that delivers the material. The supply tube can be long enough for the user to hold the supply bottle 92 in the same hand as the one that holds device 2. One example is to scrape the tongue and apply the antibacterial liquid prior to surgeries. The antibacterial liquid or gel can be held in a small squeeze bottle that is held in the hand with its supply tube extending down the length of handle 4 where it is frictionally received in port 90. The antibacterial liquid or gel can be supplied by a pressurized bulk supply controlled by the user. In another configuration, the liquid can be distributed via gravity through channel or channels 84 with the size of channels 84 limiting the amount of liquid applied. The application of the liquid can be controlled by tipping the device forward to scrape the tongue surface. When the user is finished, the device is pivoted to an upright position where the liquid falls back into its supply bottle. In a situation where devices 2 are disposable and intended for one-time use with patients, the liquid antibacterial material or medicine can be held in a chamber on top of head 6 and applied when device 2 is used and then discarded.

In the configuration of FIG. 6, an intermediate supply device 94 is disposed between first and second valves 96. Device 94 is filled from supply 92 through the first valve 96. This is achieved by applying pressure to supply 92 or with a pump. When the user is ready to dispense the material onto the tongue, the user squeezes device 94 and almost all of the material held within device 94 is dispensed onto the tongue. This allows a controlled volume of material to be dispensed each time device 94 is squeezed. The controlled volume is defined by the size of the chamber defined by device 94. In another configuration, an electric pump is used to meter the material onto the tongue through the head of the device.

Turning to FIGS. 10-18, shown is an embodiment of the device 2 includes a medical reservoir 210 and dispensing vents 220. The dispensing vents 220 may serve as an outlet through which a material is dispensed onto the tongue. As described above, the material is, for example, an antibacterial material such as chlorhexidine gluconate, carbamide peroxide, or a mixture of hydrogen peroxide and water; each provided as a liquid or a gel. The medical reservoir 210 may hold a pre-measured amount of a medicinal substance to be released by capillary action through the dispensing vents 220. The dispensing vents 220, in turn, may allow for capillary release of the medicinal substance. The medical reservoir 210 and dispensing vents 220 may generally comprise the same or similar curved shape. The curved shape of the medical reservoir 210 and dispensing vents 220 may further generally comprise the same or similar curved shape as the rounded edge of the end of the head 6, from the maximum width 32 to the outermost end 42. The medical reservoir 210 and dispensing vents 220 may be defined by head 6 and disposed adjacent the working edge 50. In an embodiment, the scraping wall 54 and inner surface 56 of scraping wall 54 may define the working edge 50 of the medical reservoir 210 such that the back or other side of the scraping wall 54 and/or inner surface 56 may comprise the interior wall of the medical reservoir 210. The remaining walls or edges of the medical reservoir 210 may be defined by the rounded head 6 of the device 2, including the area of the head 6 defined by the maximum width edge 32 to the outermost end 42.

The medical reservoir 210 and dispensing vents 220 may be defined by head 6 and disposed adjacent the trough 60 and near inlet 40. This location may allow the material in the medical reservoir 210 to be released through the dispensing vents 220 and applied to the tongue shortly before being removed by the vacuum when the vacuum is turned on. In this position, the user can draw device 2 across the tongue once or twice with the vacuum on to remove thick debris and loosen the papillae of the tongue. Capillary action that results from this movement of the device 2 may then prompt the material in the medical reservoir 210 to be released through the dispensing vents 220. It is also noted that such dispensing of the material in the medical reservoir 210 may also be actuated by the user or other means as described herein. As the user uses the device 2 to brush or contact the tongue, the material in the medical reservoir 210 may be released through the dispensing vents 220.

Further, the device 2 may include a first elongated member 222 and a second elongated member 224 that when attached together may form the device 2 including the head 6, and handle 4. The first elongated member 222 and second elongated member 224 may each be formed of a generally continuous material and each define a portion of the device 2. The first elongated member 222 may include a portion of the cavity 16 and the second elongated member 224 may include the remaining portion of the cavity 16 such that once they are attached the cavity 16 is completed to allow for the device 2 to function as described herein. In an embodiment, the first elongated member 222 may include a portion of the head 6 that includes the dispensing vents 220, the wall 54, and a trough aperture 60′ while the second elongated member may include a portion of the head 6 that includes the medical reservoir 210, an alignment wall 54′, and the trough 60. Once the members are attached, the inlet 40 is formed to allow the trough 60 to communicate with the inlet 40 and cavity 16 while also aligning the dispensing vents 220 with the reservoir 210, aligning the trough aperture 60′ with the trough 60, and aligning the wall 54 with the alignment wall 54′. Notably, a liquid or material may be placed in the reservoir 210 prior to attaching the first elongated member 222 to the second elongated member 224 when making the device 2.

The function of the device 2 is to disrupt, capture, and remove biofilm from the oral cavity. In an embodiment, the head 6 of the device 2 has a reservoir 210 and a vacuum chamber 16 trailing the reservoir 210. The reservoir 210 may be filled with a metered, therapeutic amount of chlorhexidine (CHX), for example. As noted, however, the reservoir 210 may be filled with a variety of other materials and substances as well. The head 6 may include vertical fins or vents 220 at the surface of the reservoir 210. The CHX is released by way of capillary action and surface tension. As the head 6 is glided along tissue, it releases a film of CHX and vacuums both biofilm and biofilm contents from the surface of the tissue. The design, location and function of the reservoir 210 assures that the CHX is not aspirated into the respiratory tract. The biofilm that is being removed may contain optochin sensitive bacteria such as Streptococcus pneumonia, that, when aspirated, accelerates the risk of pneumonia.

The dispensing vents 220 may include anywhere from 1 to 30 dispensing vents. The dispensing vents 220 may be of any suitable shape, including circular, rectangular, square, ovular, irregular, etc. The dispensing vents 220 may be of varying sizes and shapes or of varying sizes and shapes. In an embodiment, the dispensing vents 220 may include 10-20 dispensing vents 220 arranged adjacent and linearly to one another, where the dispensing vents 220 are each similar in shape insofar as an elongated rectangular shape, but where the dispensing vents 220 increase and decrease sequentially in length to the curvature of the rounded end of the device. This embodiment is shown in FIG. 12 for example. In another embodiment, the dispensing vents 220 may include 10-20 dispensing vents 220 arranged adjacent to one another, where the dispensing vents 220 are each similar in shape insofar as an elongated rectangular shape and where the dispensing vents 220 are substantially the same in length, but where the positioning of each dispensing vent 220 to one another is on a curve. This embodiment is shown in FIGS. 11B and 11C for example.

The application of the material can be done with the vacuum on or the vacuum off. This applied material may then be removed from the surface, such as the surface of the tongue, before it is swallowed or aspirated. The applied material may be removed by the working edge 50 of the device 2 and a brushing motion and/or the suction created by the vacuum when the vacuum is turned on. When the material was applied with the vacuum off, another pass of device 2 on the tongue with the vacuum turned on removes the material. When applied with the vacuum on, the material is removed quickly leaving only a trace functional amount or a thin coating of the material on the papillae.

In another embodiment as illustrated by FIG. 16, it may be desirable to include a brush 310 having a plurality of bristles 312. Such brush 310 may be provided on the opposite head surface 6′ of the device which is on the opposite side of the “working surface” and trough 60. The brush may utilized before or after suction or brushing by the working edge 50. If utilized before suction, the brush 310 may facilitate initial debris collection and accumulation. If utilized after suction, the brush 310 may facilitate final cleaning of a surface within the mouth, such as the tongue, teeth, or gums. The brush 310 may also be utilized after dispensing of a material from the medicinal reservoir 210 to, for example, activate or apply the material to a part of the mouth.

The brush may be arranged with various shapes along the head surface 6′ and this disclosure is not limited to the configuration of the brush and bristles. FIG. 16A illustrates the brush 310 having an elongated shape that extends from an edge of the head surface 6′ along the head towards the handle. The plurality of bristles 312 may comprise anywhere from 2-100 bristles of any varying circumference and height. In an embodiment, each bristle may be of the same circumference and height or varying circumferences and heights. The bristles 312 may comprise any suitable material including synthetic fibers such as nylon, natural materials such as bamboo viscose, or any other flexible polymers or fibers as may be desired. Each “bristle” 312 may actually include a plurality of fibers arranged in a single tuft or each bristle 312 may include a single elongated structure as shown in FIG. 16A. The bristles 312 may also be integral to a brush plate 313 that is attached to the surface of the device 2.

In the embodiment of FIGS. 16B-16D, the brush 310, brush plate 311, and bristles 312 may have a perimeter shape that is generally complimentary to the shape of the head surface 6′. This perimeter shape may include a first straight edge 315 that intersects with a second straight edge 316 along an apex 317 that is positioned towards the handle and a rounded edge 318 that aligned along a top rounded edge 7′ of the head surface 6′. This perimeter shape of the brush 310 was found to be efficient and practical use of space along the head surface 6′ as well as effective to allow for the The plurality of bristles 312, in an embodiment, may further be connected to the medicinal reservoir 210 so that release of the material in the medicinal reservoir 210 can occur through either or both the bristles 312 on the back end of the head 6 or through the dispensing vents 220 on the “working surface” of the device. In an embodiment, the device 2 may include two separate but adjacent medicinal reservoirs so that the contents of one medicinal reservoir may be dispensed through the dispensing vents 220 and the contents of the other medicinal reservoir may be dispensed through the bristles 312 or brush 310 surface.

FIGS. 17A, 17B, 18A, and 18B generally illustrate an embodiment of the device 2 wherein the first elongated portion 222 is attached to the second elongated portion 224. Here, the handle 4 and the head 6 are positioned along an axis such as Axis A shown in FIG. 17B. As a result, device 2 may generally be straight and trough 60 and vents 220 may face away from or generally normal to the Axis A and away from the handle 4 and head 6. In an embodiment, the wall defining the area between trough 60 and reservoir 210 and adjacent the working edge 50 may be generally normal and the inlet 40 which is opposite the wall across trough 60 is configured to remove debris from the trough 60 into the vacuum passage 16. It is the working edge 50, which is also not handle-facing and faces away from or generally normal to the Axis A relative to the device, that is configured to direct debris into the trough.

Turning to FIGS. 20A-25, the body of head 6 disposed in front of trough 60 and working edge 50 includes a recess 90 that is configured to hold a material that is applied to the tongue that can be applied before or after the scraping of the tongue. Recess 90 may be larger in volume than trough 60 and has ends disposed behind the ends of trough 60. Some materials to be applied to the tongue can be held within recess 90 based on their own viscosity and surface tension. Other materials are held by a material holder 92 disposed in recess 90. An example of material is a sponge that is secured in recess 90 by an adhesive. Material holder 92 can be disposed down below surface 52 so that it readily contacts the surface of the tongue to apply the material held by holder 92.

In an embodiment, material holder 92 is compressible and resilient so that it can be compressed down into recess 90 and sealed with a seal 94 that is removed prior to use. Seal 94 prevents the material carried by holder 92 from drying. Seal 94 can be applied with an adhesive or can be in the form of a cap that snaps onto head 6. When seal 94 is removed, holder 92 expands. Prior to being sealed, the material to be applied to the tongue is added to holder 92. The material is chlorhexidine, chlorhexidine gluconate, carbamide peroxide, a mixture of hydrogen peroxide and water, or another suitable material that functions as a disinfectant or an antiseptic. Holder 92 is configured to prevent the material from simply falling out of head 6 but allows the material to be applied to the tongue through contact between the tongue and holder 92. The material can be added to holder 92 as a liquid, a gel, or a powder. In other configurations, a gel or solid piece of the material is simply carried by recess 90. Seal 94 has a loose tab 96 that allows the user to readily remove seal 94. Seal 94 is patterned or colored different from head 6 so that the user can readily determine if seal 94 is in place. Using recess 90 and holder 92 limits the amount of material that can possibly be applied to the tongue. It also eliminates the risk of spraying the material into the patient's throat.

Head 6 can be fabricated from a material that transmits light such that a light source can be embedded in head 6 to illuminate the body of head 6 when the light is turned on. The light source can be an LED 100 disposed in head 6 itself or disposed back along handle 4 with a light rod or light fibers 102 to direct the light to head 6. FIG. 19E shows both options. The power for light 100 can be carried by device 2 or supplied from an outside source.

In another configuration of device 2 depicted in FIGS. 21-25, the body of head 6 disposed in front of trough 60 and working edge 50 defines a reservoir or recess 90 that is configured to hold a liquid material that is applied to the tongue immediately after the scraping of the tongue. Some materials to be applied to the tongue can be held within recess 90 based on their own viscosity and surface tension. Other materials are held by a material holder 192 disposed in recess 90. Material holder 192 may be disposed within recess 90 above bottom surfaces 52 and 193 (FIG. 25) or flush with bottom surfaces 52 and 193 so that it readily contacts the surface of the tongue to apply the material held by holder 192. In the embodiments of FIGS. 21-25, material holder 192 includes a web of internal walls 194 that extend between front 196 and rear 198 walls to define a plurality of openings 200. Openings 200 have a width that retains the material to be applied to the tongue through surface tension. Exemplary openings 200 have a width of between 0.01 to about 0.1 inches and more particularly about 0.034 inches with internal walls 194 having a width of about 0.001 inches to about 0.1 inches and more particularly about 0.016 inches.

In the one embodiment, the internal walls 194 may be in a generally parallel arrangement relative to one another while also extending between the front 196 and rear 198 walls along the curves of the head. In other configurations, walls 194 cross each other as in “X” shapes or “+” shapes. Openings 200 also can be circular or oval. Internal walls 194 may be shorter than front 196 and rear 198 walls to define a common chamber 202 above internal walls 194 that is a subchamber of recess 90. The volumes of these items are depicted in FIG. 24. In another configuration, openings 200 are individually defined by the head 6 with no common chamber connecting openings 200 with a total volume of all openings 200 being sufficient to hold the material. Material holder 192 retains a known quantity of material to be applied to the tongue so that the user of the device knows the maximum volume of material that has been applied. In the exemplary configuration, a maximum of five drops of material (about 0.2458 ml) of material can be retained by holder 192 and then applied to the tongue.

Material holder 192 may be formed from a polymer. Material holder 192 can be formed separate from head 6 and held in place by an adhesive, a press or snap fit, or by molding it in place. In another configuration, material holder 192 can be integrally molded with head 6 such as the reservoir of FIGS. 11B and 11C.

A seal 94 (shown in FIG. 23) may be removed prior to use. Seal 94 prevents the material carried by holder 92 from drying up or leaking through during storage or transport. Seal 94 can be applied with an adhesive or can be in the form of a cap that snaps onto head 6. Prior to being sealed, the material to be applied to the tongue may be added to chamber 202. The material may be chlorhexidine, chlorhexidine gluconate, carbamide peroxide, a mixture of hydrogen peroxide and water, or another suitable material that functions as a disinfectant or an antiseptic. Holder 192 may be configured to prevent the material from simply falling out of head 6 but also allow the material to be applied to the tongue through contact between the tongue and the material retained by holder 192. The material can be added to holder 192 as a liquid or a gel. The spacing between internal walls 194 may be configured to cause the surface tension of the material to retain the material until it is brought into contact with the tongue. At that time, the material is released onto the tongue by gravity and contact with the tongue. No squeezing of holder 192 is required to dispense the material. Seal 94 is patterned or colored different from head 6 so that the user can readily determine if seal 94 is in place. Using recess 90 and holder 192 limits the amount of material that can possibly be applied to the tongue. It also eliminates the risk of spraying the material into the patient's throat.

Another feature that can be included with any of the above configurations is the addition of a light 110 disposed at head 6. In FIGS. 1-3, light 110 is provided by a small light emitting diode (LED) powered by a battery 112 and controlled with a switch 114. In this configuration, switch 114 is disposed at head 6. Switch 114 can be located at other locations along handle 4. Light 110 can be disposed at the front of wall 54 to shine down on the tongue.

It is noted that although particular structural features may be described or shown in regard to a single embodiment, that these structural features may be applied to any other embodiment described herein without departing from the disclosure.

Although the embodiments of the present teachings have been illustrated in the accompanying drawings and described in the foregoing detailed description, it is to be understood that the present teachings are not to be limited to just the embodiments disclosed, but that the present teachings described herein are capable of numerous rearrangements, modifications and substitutions without departing from the scope of the claims hereafter. The claims as follows are intended to include all modifications and alterations insofar as they come within the scope of the claims or the equivalent thereof

What is claimed is:

1. A tongue cleaning device, comprising:
a elongated body including a head and a handle, including a vacuum passage that extends therein, wherein the vacuum passage facilitates suction of debris through the elongated body;
a working surface on a first side of the head, the working surface including:
an inlet that is fluidly connected to the vacuum passage and that is adjacent a working edge, wherein the working edge is configured to scrape a surface of the tongue;
a trough adjacent the working edge and in communication with the inlet; and
a reservoir positioned on the head distal to the inlet that including a plurality of dispensing vents;
wherein the reservoir is configured to hold a substance that configured to be released through the dispensing vents when in use.

2. The tongue cleaning device of claim 1, wherein the substance is an antibacterial agent, a medicine, or a freshener agent.

3. The tongue cleaning device of claim 2, wherein the substance is chlorhexidine gluconate, carbamide peroxide, a mixture of hydrogen peroxide and water.

4. The tongue cleaning device of claim 1, wherein the inlet is aligned on an axis defined by the handle.

5. The tongue cleaning device of claim 4, wherein the inlet faces away from the handle.

6. The tongue cleaning device of claim 1, wherein the working edge comprises a wall that separates the inlet from the reservoir.

7. The tongue cleaning device of claim 6, wherein the working edge is aligned generally normal to the axis defined by the handle.

8. The tongue cleaning device of claim 1, wherein the working edge is configured to direct debris into the opening.

9. The tongue cleaning device of claim 1, further including a brush on a second side of the head, wherein the brush includes a plurality of bristles.

10. The tongue cleaning device of claim 9, wherein the brush includes a perimeter shape that includes a first straight edge that intersects a second straight edge at an apex positioned towards the handle and a rounded edge that is aligned with a top rounded edge of the head.

11. A tongue cleaning device comprising:
a first elongated member that includes a head portion and a handle portion; and
a second elongated member that includes a head portion and a handle portion;
wherein when the first elongated member is attachable to the second elongated member to form a head in communication with a cavity wherein the head includes a reservoir or recess configured to dispense a substance and a trough configured to collect a substance to be vacuumed through the cavity.

12. The tongue cleaning device of claim 11 wherein the handle portion of the first elongated member includes a first portion of the cavity and the head portion of the first elongated member includes a plurality of dispensing vents and a trough aperture.

13. The tongue cleaning device of claim 12 wherein the handle portion of the second elongated member includes a second portion of a cavity and the head portion of the first elongated member includes at least a portion of the reservoir and a trough.

14. The tongue cleaning device of claim 11 further comprising an inlet between the first elongated portion and the second elongated portion to allow the trough to communicate with the cavity.

15. The head of claim 12, wherein the plurality of dispensing vents are configured to release the substance from the reservoir.

16. The tongue cleaning device of claim 11 further comprising a wall positioned between the reservoir and the trough such that the trough is configured to collect material from a surface of the tongue to withdrawn through the cavity.

17. The tongue cleaning device of claim 11 wherein the first elongated member and the second elongated member are attached along an elongated axis that is generally parallel to the axis of the cavity.

18. The tongue cleaning device of claim 11 wherein the first elongated member and the second elongated member are each formed of a common continuous material.

19. The tongue cleaning device of claim 11, further including a brush on a second side of the head, wherein the brush includes a plurality of bristles.

20. The tongue cleaning device of claim 19, wherein the brush includes a perimeter shape that includes a first straight edge that intersects a second straight edge at an apex positioned towards the handle and a rounded edge that is aligned with a top rounded edge of the head.
